(19) United States
(12) Patent Application Publication
(10) Pub. No.: US 2024/0423504 A1
(43) Pub. Date: Dec. 26, 2024

(54) MEASUREMENT PROCESSING TERMINAL, METHOD, AND COMPUTER PROGRAM FOR PERFORMING MEASUREMENT PROCESSING OF MOTION OF FINGER

(71) Applicant: MAXELL, LTD., Tokyo (JP)

(72) Inventors: Yoshiharu UCHIDA, Kyoto (JP); Tomohiko MIZUGUCHI, Kyoto (JP)

(21) Appl. No.: 18/691,853

(22) PCT Filed: Sep. 16, 2021
(86) PCT No.: PCT/JP2021/034132

§ 371 Date: Mar. 13, 2024

(51) Int. Cl. A61B 5/11; A61B 5/00; A61B 5/16

(52) U.S. Cl. CPC A61B 5/1125; A61B 5/1114; A61B 5/1128; A61B 5/163; A61B 5/4088

(57) ABSTRACT

A measurement processing terminal includes: an imaging data collector that collects imaging data obtained by capturing a motion of a finger of a subject; a hand tracking data generator that implements a hand tracking function of detecting and tracking a position of the finger on the basis of the imaging data and generates chronological hand tracking data by the hand tracking function from the imaging data; and a data processor that processes the hand tracking data obtained from the hand tracking data generator to generate quantitative data regarding the bending/stretching motion of the finger and/or the opening/closing motion of two fingers accompanying the motion of the finger joint, and processes the hand tracking data, distance data and time data obtained from a movement distance measuring instrument, and line-of-sight data obtained from a gaze detector to generate correlation data obtained by quantifying a correlation between these data.

### TECHNICAL FIELD

The present invention relates to a measurement processing terminal, a measurement processing method, and a computer program that measure a motion of a finger including a finger tapping motion or the like and process the measurement result.

### BACKGROUND ART

With the aging of society, the number of patients with Alzheimer's dementia is increasing year by year. If patients are detected early, medication can slow the progression of the disease. Since it may be difficult to distinguish between symptoms associated with aging such as forgetfulness and the disease, there are many cases where patients go to a hospital only after the disease becomes severe.

In such a situation, as a screening test for early detection of Alzheimer's dementia, a blood test, a smell test, a test that reproduce doctor's interviews on a tablet terminal and the like have been conventionally performed. But there has been problems such as pain during blood sampling and long examination times, which places a heavy burden on the subject. On the other hand, as an examination with a small burden on the subject, cognitive function evaluation is also performed by button pressing or finger motion measurement of one hand using a tablet terminal, but there is a problem that sufficient examination accuracy cannot be obtained. If a simple screening examination can be performed with high accuracy with a small burden on the subject, it leads to early detection of Alzheimer's dementia, and can also contribute to improvement of the quality of life of the patient and reduction of medical and care costs.

On the other hand, in recent years, it has been revealed that a motion pattern peculiar to Alzheimer's dementia can be extracted from an opening/closing motion (finger tapping motion) of thumbs and index fingers of both hands, and it has been confirmed that there is a high correlation with the dementia examination by motion measurement of the fingers and general interview. These are said to be results of capturing a decrease in the rhythm motion function of fingers of both hands caused by atrophy of the brain in Alzheimer's dementia by finger tapping motion measurement. In addition, a finger is said to be a second brain, and many regions in the brain are related to the function of the finger, and the motion of the finger is not limited to Alzheimer's dementia, but is also said to be related to dementia such as cerebrovascular disease, Lewy body disease, Parkinson's disease, developmental coordination disorder (such as inability to skip or jump rope), and the like. That is, the state of the brain can be known from the tapping motion of the finger. Furthermore, since an ability of finger dexterity can be quantified by utilizing the tapping motion of the finger as a “measure” indicating the health condition of the brain, the tapping motion of the finger can be used in various fields such as healthcare, rehabilitation, and life support.

As a method for accurately measuring and evaluating a finger tapping motion, for example, Patent Literature 1 and Patent Literature 2 disclose a motion function evaluation system and a method including a motion function measurement apparatus that calculates motion data based on a relative distance between a pair of a transmission coil and a reception coil attached to a movable part of a living body, and an evaluation apparatus that evaluates a motion function of the living body based on motion data received from the motion function measurement apparatus. That is, these patent documents show that a state of brain function is known by converting a change in magnetic force fluctuated by tapping motion by two fingers into an electrical signal using a magnetic sensor attached to a fingertip, measuring and quantifying the motion, and capturing a feature amount indicating the feature of the finger motion.

In addition, in the rehabilitation field, Simple Test for Evaluating Hand Function (STEF) is also conducted to confirm the effect of postoperative rehabilitation of stroke and cerebral infarction patients by measuring the time required for a series of gripping and moving objects of different sizes and shapes with a stopwatch.

### CITATION LIST

### Patent Literature

Patent Literature 1: JP 2016-49123 A
Patent Literature 2: JP 2015-217282 A

### SUMMARY OF INVENTION

### Technical Problem

However, in a finger tapping apparatus using a magnetic sensor attached to a fingertip as disclosed in Patent Literature described above, since a motion of a joint of a finger cannot be recognized, a bending/stretching motion of the finger such as a “pinching” motion cannot be quantitatively evaluated. In addition, in a case where it is difficult to attach the sensor due to injury, deformation, or the like of the finger, measurement cannot be performed.

In addition, in the Simple Test for Evaluating Hand Function (STEF) described above, since a motion of an arm and fingers is performed by visual observation by a doctor, it is largely subject to a subjectivity of a measurer (doctor), and the examination results may vary depending on the measurer.

In addition, in order to objectively, finely and accurately evaluate a motor function of upper limb, it is necessary to perform an evaluation in synchronization with a motion of not only the finger but also other parts of a body such as the arm and the eye (For example, the interlocking property (relationship) of the motion of the finger and the arm and the interlocking property (relationship) of the motion of the finger and the eye are quantitatively evaluated.). However, in a method of visually confirming the motion as in a conventional examination method, it is difficult for the measurer to simultaneously evaluate the motion of different parts.

Furthermore, in order to accurately grasp the degree of recovery of the motor function of upper limb, or in order to prevent variations in an examination and measurement, it is also necessary to unify the examination and measurement environment to make the examination and measurement conditions uniform. Depending on the examination and measurement method, if such examination and measurement conditions (or environment) are not unified, the examination and measurement results vary among subjects, and accurate examination and measurement may not be performed.

The present invention has been made in view of the above circumstances, and an object of the present invention is to provide a measurement processing terminal, a method, and a computer program capable of not only quantitatively evaluating a bending/stretching motion function of a finger and/or an opening/closing motion function of two fingers by capturing a motion of a finger joint, but also objectively and finely evaluating the motor function of upper limb with high accuracy.

### Solution to Problem

In order to solve the above problems, the present invention is a measurement processing terminal that measures a motion of a finger of a subject and processes a measurement result, the measurement processing terminal including: an imaging data collector that collects imaging data obtained by capturing the motion of the finger of the subject; a hand tracking data generator that implements a hand tracking function of detecting and tracking a position of the finger on the basis of the imaging data and generates chronological hand tracking data by the hand tracking function from the imaging data; and a data processor that processes the hand tracking data obtained from the hand tracking data generator and generates quantitative data regarding a bending/stretching motion of the finger and/or an opening/closing motion of two fingers accompanying the motion of the finger joint.

According to the above configuration of the present invention, the chronological hand tracking data can be generated from the imaging data obtained by capturing the motion of the finger of the subject by the hand tracking function, and the hand tracking data can be processed to generate quantitative data regarding the bending/stretching motion of the finger accompanying the motion of the finger joint. Therefore, it is possible to accurately capture the motion of the joint that cannot be recognized by a conventional magnetic sensor type apparatus, and quantitatively evaluate the bending/stretching motion of the finger such as a “pinching” motion. As a result, in evaluation of an ability of finger dexterity, more detailed analysis and evaluation can be performed by combining the information of distance between two fingers and the information of movement (joint angle) of each joint of the finger.

Note that the measurement processing terminal having such a function may have any form. For example, the measurement processing terminal may be configured as a small terminal such as a smartphone, may be in the form of a tablet-type thin computer, a personal computer, or the like, or may be in the form of a head mounted display (Head Mounted Display; HMD, hereinafter referred to as HMD) or the like.

Furthermore, in the above configuration, the data processor may calculate and analyze a feature amount leading to brain function evaluation of the subject. In addition, the data processor may evaluate (for example, by comparison with data of a healthy subject,) brain function and cognitive function of the subject from the calculated feature amount. Such assessment may be effective as an early stage screening to discriminate dementia and may aid in the detection of dementia. In addition, the application of the measurement processing terminal with such a data processor is not limited to the clinical field, and for example, the measurement processing terminal can also contribute to determination of judgment in driving a car, can be applied to a brain training-like game and the like, and has a wide application range.

Furthermore, in the above configuration, a movement distance measuring instrument that measures a chronological movement distance in which a hand moves with a motion of an arm, or a gaze detector that detects a line-of-sight of an eye of the subject may be further provided. In this case, the data processor preferably processes the hand tracking data obtained from the hand tracking data generator, distance data and time data obtained from the movement distance measuring instrument, and line-of-sight data obtained from the gaze detector, and generates correlation data obtained by quantifying the correlation between these data. According to this, for example, when the arm is moved to grip the object, the motion of the arm and the opening/closing motion of the finger can be quantitatively evaluated at the same time (interlocking property (relationship) between the motion of the finger and the motion of the arm can be quantitatively evaluated), and furthermore, the interlocking property (relationship) between the motion of the finger and the motion of the eye can be quantitatively evaluated. That is, the finger can be evaluated in synchronization with the motion of another part of the body, and thus the motor function of upper limb can be objectively, finely and accurately evaluated.

As the correlation data generated by the data processor, for example, graph data indicating the relationship between the movement distance of the hand from the measurement start position to the object to be gripped and the time together with the opening/closing timing of the finger can be exemplified. Such correlation data makes it possible to grasp the movement distance of the hand when the subject tries to open the finger, the distance from the measurement start position to the object, and the time required to grip the object. In addition, graph data indicating the relationship between the distance between two fingers and time can be cited as correlation data. Such correlation data makes it possible to grasp the opening/closing timing of the finger (the moment of gripping the object). Furthermore, examples of the correlation data include graph data indicating a relationship between a joint angle and time obtained from the hand tracking data and the relationship between the distance from the measurement start position and the distance between two fingers. The graph data indicating the relationship between the joint angle and time makes it possible to know the change in the joint angle with time, and the graph data indicating the relationship between the distance from the measurement start position and the distance between two fingers makes it possible to know how far the hand has moved from the measurement start position before the finger opening action is performed. Furthermore, in order to be able to evaluate the motion of the finger in synchronization with the motion of the eye detected by the eye tracking of the gaze detector, for example, correlation data that can display the deviation of the line-of-sight position of the eye with respect to the object to be gripped as a scatter diagram may be generated.

In the above configuration, the data processor may generate image data related to a measurement reference position and/or a measurement history. According to this, measurement conditions can be made uniform among a plurality of examinations and measurements, and the change in the motor function of a finger (upper limb) can be known among the plurality of examinations and measurements. Note that examples of the image data related to the measurement reference position include image data for marking the measurement start position (such as a position to place a hand) on a display screen, and image data for performing guide display indicating a contour of the finger that defines a direction and a position of the hand at the time of starting measurement on the display screen. Examples of the image data related to the measurement history include image data for displaying a past measurement result on the display screen with a dotted line, a contour of the hand, or the like.

In the above configuration, a condition setter that sets the measurement condition may be further provided, and in this case, the data processor preferably generates image data and/or audio data corresponding to the measurement condition set by the condition setter. According to this, by setting the measurement conditions, for example, measurement error (variation) can be reduced, examination and measurement environment can be unified, and the examination and measurement conditions can be made uniform (unified). As a result, problems unique to the case of using a photography data are solved (For example, in consideration of a parameter that can vary as the distance from the camera as an imaging unit to an imaging target changes, references are made uniform in the Z-axis direction, and the like.), external factors (noise and the like) are reduced as much as possible, or a plurality of subjects can perform measurement under the same environment as much as possible. Therefore, for example, it is possible to accurately know the degree of recovery of the motor function of upper limb or to prevent variation in the examination and measurement states among the subjects. In addition, if the set conditions are visualized by image or made audible by voice, the measurement conditions are reliably recognized by the subject (Alternatively, external factors that adversely affect the measurement are eliminated.), an appropriate measurement environment is prepared, and an accurate measurement result can be obtained.

Here, the measurement conditions set by the condition setter may be associated with the measurement environment and the subject's field of view, in which case the data processor preferably generates audio data that eliminates noise in the measurement environment and/or image data that limits the subject's field of view. Examples of the audio data for eliminating noise in the measurement environment include audio data for outputting music for canceling surrounding noise from a speaker, and examples of the image data for limiting the subject's field of view include image data for inserting a virtual object between the finger and a surrounding object and hiding the object on the display screen.

In addition, the measurement condition set by the condition setter may be associated with the initial setting of the measurement. Examples of such initial setting include setting of a measurement reference position (positions of the head, the finger, and the line-of-sight of the subject, and the like) and setting of an acquisition position of imaging data (for example, the position of the camera) that is first performed for each measurement when measurement is executed for the same subject a plurality of times in order to accurately grasp the degree of recovery of the motor function of upper limb. In addition, such condition setting can also be performed as preprocessing of measurement processing (examination), and can contribute to unification of measurement conditions.

Furthermore, in the above configuration, the imaging data collector may include a camera that captures the motion of the finger. Such a camera can capture the measurement environment. In the above configuration, the measurement processing terminal may further include an output interface that outputs data generated by the hand tracking data generator and/or the data processor. Examples of such an output interface include a display apparatus that displays an image, text, or the like, and an audio output apparatus such as a headphone or a speaker.

In addition to the measurement processing terminal described above, the present invention also provides a method and a computer program for measuring a motion of a finger and processing a measurement result.

### Advantageous Effects of Invention

According to the present invention, a hand tracking function is implemented, and hand tracking data, movement distance data of a finger, and time data are processed to obtain correlation data obtained by quantifying correlation of these data. Therefore, not only a motion of a finger joint can be captured and a bending/stretching motion function of a finger and/or an opening/closing motion function of two fingers can be quantitatively evaluated, but also a motor function of upper limb can be objectively and finely and accurately evaluated.

### BRIEF DESCRIPTION OF DRAWINGS

FIG. 1 is a block diagram illustrating a configuration example of a measurement processing terminal as an HMD according to a first embodiment of the present invention.

FIG. 2 is a schematic view illustrating a state in which a finger tapping motion is captured by a camera of the measurement processing terminal as the HMD of FIG. 1 worn on a head of a subject.

FIG. 3 is a schematic view illustrating a state in which a hand tracking image in which a finger landmark display is superimposed on a video of a finger by a hand tracking function and numerical data of a joint angle obtained by processing of hand tracking data are displayed on an HMD screen.

FIG. 4 is a schematic view illustrating a state in which a guide display indicating a contour of a finger that defines a direction and a position of a hand at the start of measurement is displayed on an HMD screen.

FIG. 5 is a schematic view illustrating a state in which a guide display indicating a contour of a finger that defines a direction and a position of a hand at the time of starting measurement and a dotted line indicating a past measurement result (finger lifting state, finger opening state) are displayed on an HMD screen.

FIG. 6 is a schematic view illustrating a state in which a subject places his/her hand at a measurement start position in execution of an examination in which an object placed at a distant position is gripped with a hand.

FIG. 7 (a) is a schematic diagram illustrating a state in which a camera of an HMD reads a position of a hand of a subject with the subject's hand placed at a measurement start position. (b) is a schematic diagram illustrating how the camera constantly tracks the position of the hand even when the subject changes a line-of-sight within a capture range of the camera of the HMD.

FIG. 8 is a schematic view illustrating a state in which a subject moves his/her arm to move his/her hand to an object placed at a distant position.

FIG. 9 is a schematic view illustrating a state in which the subject holds an object placed at a distant position with a hand.

FIG. 10 is a graph (correlation data) illustrating the relationship between the movement distance of the hand from the measurement start position to the object to be gripped and the time together with the opening/closing timing of the finger.

FIG. 11 is a graph (correlation data) showing a relationship between a distance between two fingers and time.

FIG. 12 is a graph (correlation data) illustrating a relationship between a joint angle and time obtained from hand tracking data.

FIG. 13 is a graph (correlation data) showing a relationship between a distance from a measurement start position and a distance between two fingers.

FIG. 14 is a schematic diagram illustrating a state in which a gaze alignment marker is displayed on an HMD screen at a time of measuring a finger tapping motion, in which (a) illustrates a state in which the entire captured image of the camera including a measurement environment is displayed as it is on the HMD screen, and (b) illustrates a state in which an image (image that restricts a subject's field of view) that shields the measurement environment except for a finger is displayed on the HMD screen.

FIG. 15 illustrates an image displayed on an HMD screen when an examination in which a subject performs a finger tapping motion without looking at his/her hand is executed, in which (a) is a display image in which the finger is shielded by transmission masking, and (b) is a display image in which a contour of the finger serving as a mark of a position where the hand is placed is superimposed on the transmission masking in the display state of (a).

FIG. 16 is a flowchart illustrating an example of the operation of the measurement processing terminal as an HMD according to the first embodiment of the present invention.

FIGS. 17 show an example of examination and measurement by a subject alone using a measurement processing terminal as a smartphone according to a second embodiment of the present invention. (a) is a schematic diagram showing a state in which a front camera of a smartphone captures a motion of a face or an eye of the subject and a rear camera of the smartphone tracks and captures a finger tapping motion of the subject, and (b) is a schematic diagram showing a state in which a position and an orientation of a hand at the time of measurement are read and registered in advance.

FIG. 18 is a schematic view illustrating a state in which online medical care by a doctor or the like is further performed using a front camera in the state of FIG. 17 (a).

FIG. 19 is a schematic view illustrating a state in which a measurer such as a doctor performs tracking and capturing of a motion of a finger of a subject with a rear camera of a smartphone.

FIG. 20 is a schematic view illustrating a state in which both a subject and a measurer share an examination and measurement image using a smartphone (foldable smartphone) having two display screens that are opened and closed.

### DESCRIPTION OF EMBODIMENTS

Hereinafter, embodiments of the present invention will be described with reference to the drawings. The present embodiment contributes to the development of medical care and the realization of a healthy society with advanced technologies by providing the following technologies. The implementation of the measurement processing terminal contributes to the “9. Build resilient infrastructure, promote inclusive and sustainable industrialization and foster innovation” of the Sustainable Development Goals (SDGs) proposed by the United Nations.

Note that, in the following, the measurement processing terminal according to the present invention that performs measurement processing of a motion of a finger will be described as a head mounted display (HMD) (first embodiment) or as a smartphone (second embodiment). However, the measurement processing terminal of the present invention may have a form such as a thin tablet computer, a personal computer, or the like, or a use form in which the measurement processing terminal is connected to a server via a communication means (network) is also conceivable, and any structure form and use form may be adopted.

In addition, in the following embodiments, a measurement processing terminal that includes a camera, a display, or the like so as to be able to acquire captured imaging data and measure and display the motion of the finger by itself is illustrated. However, the present invention may be embodied as a terminal or a method that enables measurement processing of the motion of the finger in cooperation with a separate imaging camera and a display, or may be configured as a computer program that enables such measurement processing to be performed by a computer.

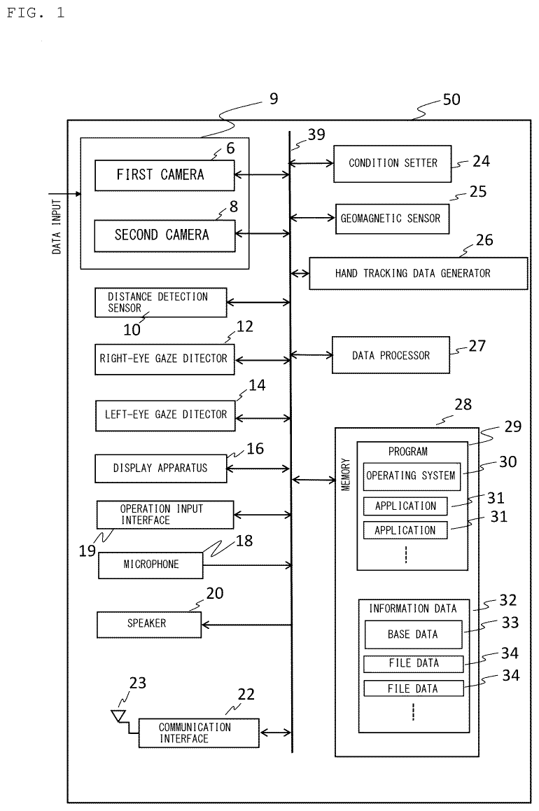

FIGS. 1 to 16 illustrate the first embodiment of the present invention in which the measurement processing terminal that measures the motion of the finger of the subject and processes a measurement result is embodied as a head mounted display (HMD) 50. FIG. 1 shows a block diagram of a configuration of such an HMD 50.

As illustrated in FIG. 1, the HMD 50 includes first and second cameras 6 and 8, a distance detection sensor 10, an optional geomagnetic sensor (gravity sensor) 25, a hand tracking data generator 26, a condition setter 24, a data processor 27, right-eye and left-eye gaze detectors 12 and 14, a display apparatus 16, an optional operation input interface 19, a microphone 18, a speaker 20, a memory 28 including a program 29 and information data 32, a communication interface 22, and a transmission/reception antenna 23, and these components are connected to each other via a bus 39 except for the transmission/reception antenna 23. In this case, at least the first and second cameras 6 and 8 and the distance detection sensor 10 constitute a measuring instrument that measures the motion of the finger of the subject. In addition, in the present embodiment, the first and second cameras 6 and 8 that capture the motion of the finger of the subject are provided, and these cameras 6 and 8 constitute an imaging data collector 9 that collects imaging data obtained by capturing the motion of the finger of the subject. However, in another embodiment, the imaging data collector 9 may capture imaging data obtained by capturing the motion of the finger of the subject by a camera separate from the measurement processing terminal via a data input interface without providing the cameras 6 and 8.

The first camera 6 is a rear camera built in the HMD 50 to capture the motion of the finger of the subject including a measurement environment (surrounding object or scene), and the second camera 8 is built in the HMD 50 as a front camera to capture an eye of the subject for eye tracking by gaze detectors 12 and 14. Each of the cameras 6 and 8 captures an image of an object and captures the captured image (imaging data).

The distance detection sensor 10 constitutes a movement distance measuring instrument that measures a chronological movement distance in which a hand moves with motion of an arm, and is a sensor capable of capturing a shape of an object such as a person or an object as a three-dimensional shape (Alternatively, a timer that measures time may be separately provided.). Examples of such a sensor include a Light Detection and Ranging (LiDAR) sensor that irradiates an object with laser light such as infrared light, measures reflected scattered light, and analyzes and detects a distance to an object at a long distance and a state of the object, a Time Of Flight (TOF) sensor that measures a reflection time of pulsed light applied to the object for each pixel and measures a distance, and a millimeter wave radar that emits a millimeter wave, captures the reflected wave, and detects a distance to the reflecting object and a state of the object. In particular, the distance detection sensor 10 of the present embodiment can detect the distance to the finger of the subject and the angle thereof, and enables chronological measurement of each distance.

The right-eye gaze detector 12 and the left-eye gaze detector 14 detect a line-of-sight of the right eye and the left eye of the subject, respectively. Note that the processing of detecting the line-of-sight may use a well-known technique generally used as eye tracking processing. For example, in a method using corneal reflection, there is known a technique of irradiating a face with an infrared Light Emitting Diode (LED) and photographing a face with an infrared camera, and detecting the line of sight on the basis of a position of a pupil with respect to a position of corneal reflection with a position (corneal reflection) of reflected light generated by irradiation of the infrared LED as a reference point.

The geomagnetic sensor 25 is a sensor (gravity sensor) that detects the magnetic force of the earth, and detects the direction in which the HMD 50 is facing (an angle of a neck of the subject). As the geomagnetic sensor, it is also possible to detect the motion of the HMD 50 (the angle of the neck of the subject) by capturing a geomagnetic change with respect to a motion of the HMD 50 using a three-axis type that also detects geomagnetism in vertical direction in addition to front-back direction and left-right direction.

The condition setter 24 is for setting a measurement condition, and includes, for example, a user interface for selecting an examination mode such as a motor function of upper limb or a finger tapping motion function or selecting measurement conditions prepared for each examination mode, and the measurement condition can be set by displaying the user interface on a display screen of the HMD 50, performing gesture operation or voice input, or performing input via an input means such as a keyboard, a key button, or a touch key. Examples of the measurement condition in the finger tapping motion function prepared as the user interface include selection of a measurement mode such as both hands at the same time, both hands alternately, only one hand (right hand), and only one hand (left hand), selection of whether or not to show a finger part to the subject at the time of measurement (on/off of the function of the transmission masking of the finger), and selection of whether or not to suppress noise relating to the measurement environment sensed by the eye and ear of the subject (for example, on/off of a function of inserting a virtual object between the finger and a surrounding object to hide surrounding information). In addition, as an example of the measurement condition prepared as another user interface, for example, a function of displaying a past measurement result or a target standard with a finger contour, a dotted line, or the like may be set so that a change (In the case of rehabilitation training, its rehabilitation effect) in the motor function of a finger (upper limb) can be grasped.

In addition, the hand tracking data generator 26 implements a hand tracking function of detecting and tracking the position of the finger based on the imaging data acquired by the camera 6, and generates the chronological hand tracking data from the imaging data by the hand tracking function. As the hand tracking (skeleton detection) function, for example, an open source machine learning tool “MediaPipe” provided by Google LLC (U.S.A.) may be utilized.

In addition, the data processor 27 not only processes the hand tracking data obtained from the hand tracking data generator 26 to generate quantitative data related to a bending/stretching motion of the finger and/or an opening/closing motion of the two fingers accompanying the motion of the finger joint, but also processes the hand tracking data obtained from the hand tracking data generator 26, the distance data and the time data obtained from the distance detection sensor 10, and the line-of-sight data obtained from the gaze detectors 12 and 14 to generate correlation data obtained by quantifying the correlation between these data. Further, the data processor 27 generates image data related to a measurement reference position and/or a measurement history, and also generates image data and/or audio data corresponding to the measurement condition set by the condition setter 24. In particular, when the measurement condition is associated with the measurement environment and the subject's field of view, the data processor 27 generates audio data that eliminates noise in the measurement environment and/or image data that limits the subject's field of view. Note that, in the present embodiment, the data processor 27 also constitutes a controller of the HMD 50, and is constituted by a CPU or the like, and executes the program 29 such as an Operating System (OS) 30 and various operation control applications 31 stored in the memory 28 to perform operation control processing of the entire HMD 50 and control startup operation of various applications.

The memory 28 includes a flash memory or the like, and stores the program 29 such as the operating system 30 and the operation control application 31 for various processes such as an image, audio, document, display, and measurement. The memory 28 also stores information data 32 such as base data 33 necessary for basic operation by the operating system 30 or the like, and file data 34 used by the various applications 31 or the like. For example, the image processing application is activated, the image is captured by the camera, and the captured file data is stored. Note that the processing in the data processor 27 may be stored as an application A, and the measurement processing of the motion of the finger and the calculation analysis of various feature amounts may be performed by activating the application A. In addition, a measurement result measured from the information processing terminal may be received by an external server apparatus or the like having high calculation performance and a large capacity, and the feature amount may be calculated and analyzed.

The display apparatus 16 is an output interface that outputs data generated by the hand tracking data generator 26 and/or the data processor 27, and can display, in particular, a processing result processed by the data processor 27. In the case of an optically transmissive HMD, the display apparatus 16 includes, for example, a projection unit that projects various types of information such as reproduction information by a start-up application and notification information to the subject, and a transparent half mirror that forms and displays the projected various types of information in front of the subject's eyes. In addition, in the case of a video transmissive HMD, the HMD includes a display such as a liquid crystal panel that displays a real space object in front of the subject captured by the first camera 6 and various types of information together. This allows the subject to visually recognize and view not only the image in the field of view in front of the subject but also image information from other sources.

Note that, in the second embodiment to be described later, since the measurement processing terminal is a smartphone, the display apparatus 16 includes a liquid crystal panel or the like, and can display notification information to the subject such as a remaining amount of battery capacity, various alarms and time, and an icon of an application to be activated in the display screen, and the like, together with display of an image and a video.

The operation input interface 19 of the HMD 50 often uses gesture operation or audio input, but for example, an input means such as a keyboard, a key button, or a touch key may be used, and information desired to be input by the subject is set and input. In the second embodiment to be described later, since the measurement processing terminal is the smartphone, the operation input interface 19 can be provided in the terminal itself. However, in the present embodiment, the operation input interface 19 may be provided at a position or in a form in which the subject easily performs an input operation in the HMD 50, or may be in a form of being connected in a wired or wireless manner separately from the main body of the HMD 50. In addition, an input operation screen may be displayed in the display screen of the display apparatus 16, and input operation information may be fetched according to the position on the input operation screen to which the line-of-sight detected by the right-eye gaze detector 12 and the left-eye gaze detector 14 is directed, or a pointer may be displayed on the input operation screen and the pointer may be operated by the operation input interface 19 to fetch the input operation information. In addition, the subject may utter a voice indicating the input operation, collect the voice with the microphone 18, and capture the input operation information.

The microphone 18 can also constitute an output interface that outputs audio data generated by the data processor 27, and collects a voice from the outside or a user's own vocalization. Furthermore, the speaker 20 outputs a voice to the outside to inform the user of a voice such as notification information or music. In addition, the instruction related to the measurement of the finger motion may be transmitted to the subject by voice through the speaker 20.

The communication interface 22 is a communication interface that performs wireless communication with a server apparatus or the like at another location by short-range wireless communication, wireless LAN, or base station communication, and transmits and receives measurement data, analyzed and calculated feature amount, and the like to and from the server apparatus or the like via the transmission/reception antenna 23 at the time of wireless communication. Note that the short-range wireless communication is performed using, for example, an electronic tag, but is not limited thereto, and may be performed using a wireless LAN such as Bluetooth (registered trademark), IrDA (Infrared Data Association, registered trademark), Zigbee (registered trademark), HomeRF (Home Radio Frequency, registered trademark), or Wi-Fi (registered trademark) as long as the measurement processing terminal can perform at least wireless communication when the measurement processing terminal is near another information terminal. Further, as the base station communication, wireless communication over a long distance such as W-CDMA (Wideband Code Division Multiple Access) or GSM (Global System for Mobile communications, registered trademark) may be used. It is also possible to detect a positional relationship and an orientation between terminals by using an Ultra Wide Band (UWB). Although not illustrated, the communication interface 22 may use another method such as communication using optical communication sonic communication and the like as a means of wireless communication. In this case, instead of the transmission/reception antenna 23, a light emitter/receiver and a sound wave output/sound wave input interface are used, respectively.

Note that, in the present embodiment, the HMD 50 individually includes the above-described respective components as described above, but may include a functional unit that integrates at least some or all of these components, and in short, may have any configuration as long as the functions of these respective components are secured.

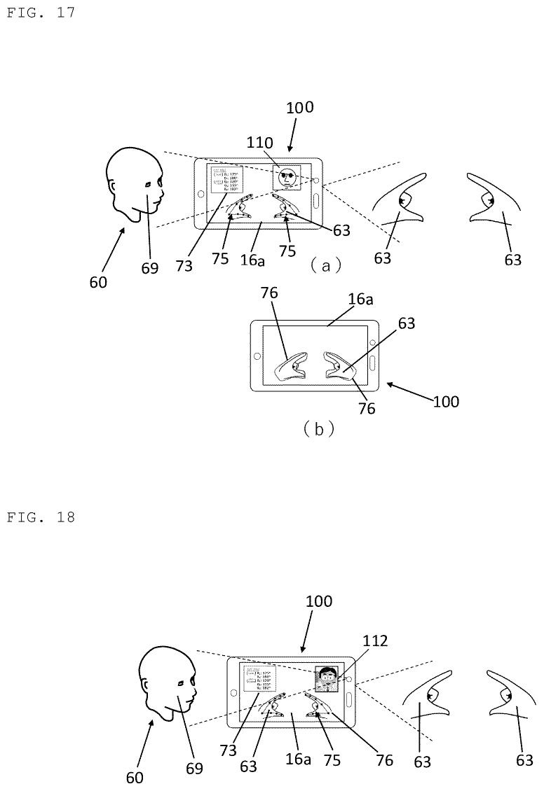

Next, the measurement processing operation of the HMD 50 in which measurement processing of the motion of the finger is performed will be described with reference to FIGS. 1 to 15 and the flowchart illustrated in FIG. 16.

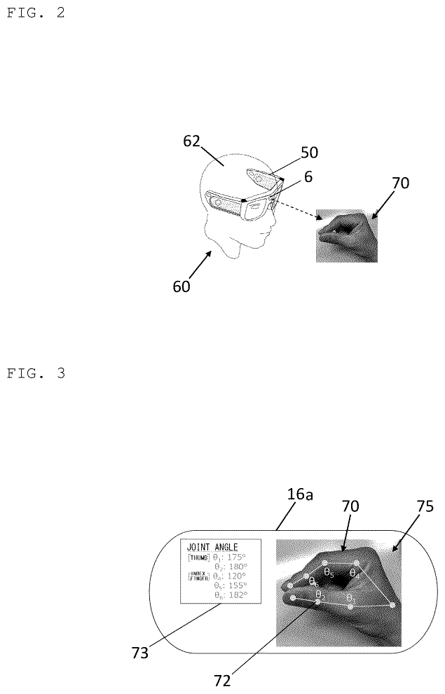

FIG. 2 illustrates a state in which the finger tapping motion performed by a subject 60 is captured by the first camera 6 of the HMD 50 mounted on a head 62 of the subject 60. On the screen of the display apparatus 16 of the HMD 50, a moving image 70 of the finger of the subject 60 captured by the first camera 6 is displayed.

FIG. 3 illustrates a state in which a hand tracking image 75 in which a finger landmark display 72 is superimposed on the moving image 70 of the finger by the hand tracking function described above, and various types of information related to measurement obtained by the data processor 27 processing the hand tracking data generated by the hand tracking data generator 26, for example, a numerical data 73 of the joint angle are displayed on a screen 16a of the display apparatus 16 of the HMD 50. Of course, since the data related to the finger landmark is essentially used in the data processing in the data processor 27, the data may not be displayed to the subject (the finger landmark display 72 may not be displayed on the screen 16a).

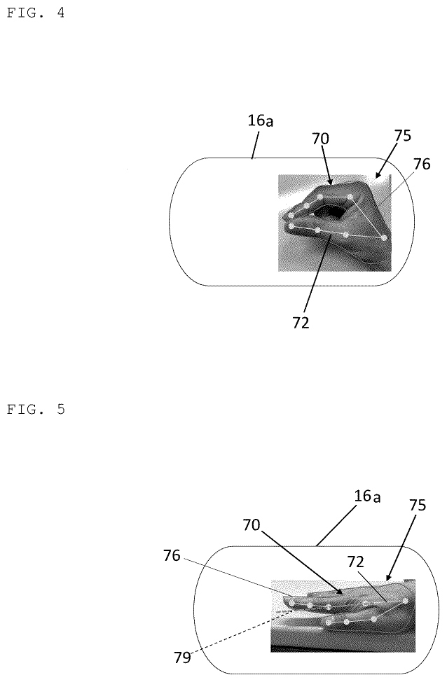

FIG. 4 illustrates a state in which, together with the hand tracking image 75, a guide display (guide contour) 76 indicating the contour of the finger that defines the direction and position of the hand at the start of measurement is displayed on the screen 16a of the display apparatus 16 of the HMD 50 as the image data related to the measurement reference position. The hand position and orientation at the time of measurement has been read and registered (stored in the memory 28) in advance through the first camera 6, and they are read out from the memory 28 for each measurement, thereby the guide display 76 is displayed on the screen 16a in the position at the time of being read. Such a guide display 76 not only forms image data related to the measurement reference position, but also can form image data for initial setting to be first performed for each measurement in a case where measurement is executed for the same subject a plurality of times in order to accurately grasp the degree of recovery of the motor function of upper limb, so that measurement conditions can be unified among a plurality of examinations and measurements. As a matter of course, the transmitted image of the hand may be displayed instead of the contour of the finger. The image data related to the measurement reference position is generated by the data processor 27.

FIG. 5 illustrates a state in which a dotted line 79 indicating the past measurement result (finger lifting state, finger opening state) is displayed on the screen 16a of the display apparatus 16 of the HMD 50 as the image data related to the measurement history together with the guide display 76 indicating the contour of the finger that defines the direction and position of the hand at the start of measurement. Of course, instead of the dotted line 79, the contour of the finger indicating the finger lifting state and the finger opening state in the past may be displayed. Such image data related to the measurement history makes it possible to grasp a change in the motor function of a finger (upper limb) (in the case of rehabilitation training, its rehabilitation effect) among the plurality of examinations and measurements. The image data related to the measurement history is generated by the data processor 27.

In addition, in a case where the HMD 50 is shared by a plurality of persons, unique identification information such as a fingerprint and a palm print may be registered in advance and collated before measurement so that the information of the contour of the finger of the subject may be read and displayed.

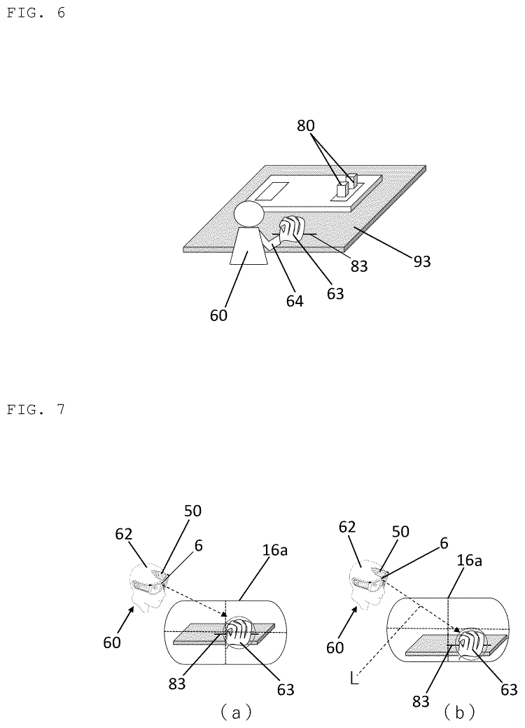

FIG. 6 illustrates a state in which the subject 60 places his/her hand 63 at the measurement start position in execution of the examination in which the subject 60 grips an object 80 placed at a distant position by hand. Such a measurement start position is displayed as a marking 83 (for example, on a desk 93 shown in an image capturing a measurement environment) on the screen 16a of the display apparatus 16 of the HMD 50 as the image data related to the measurement reference position. At the start of such an examination, it is conditioned, for example, to place the hand 63 at the position of such marking 83 (step S1 in FIG. 16). The image data related to the measurement reference position is generated by the data processor 27.

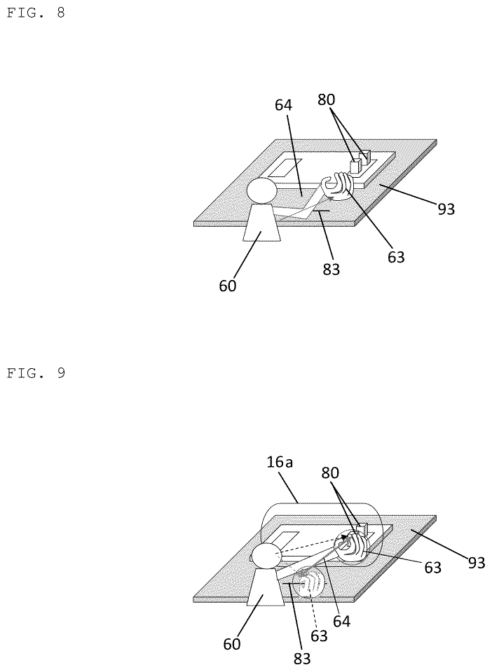

FIG. 7 (a) illustrates a state in which the first camera 6 of the HMD 50 reads the position of the hand 63 in a state in which the subject 60 places his/her hand 63 on the marking 83. At the start of the examination, such a start position is also read (step S2 in FIG. 16). FIG. 7 (b) is a schematic diagram illustrating a state in which the first camera 6 constantly tracks the position of the hand 63 even when the subject 60 changes the line-of-sight L within the capture range of the first camera 6 of the HMD 50. In this sense, it is preferable that the first camera 6 can capture the position of the hand 63 in a wide range.

When the measurement condition is set by the condition setter 24 at the start of the examination (condition setting step S3 in FIG. 16), the data processor 27 generates image data and/or audio data corresponding to the measurement condition (step S4 in FIG. 16). By setting the measurement conditions, for example, the measurement error (variation) can be reduced, the examination and measurement environment can be unified, and the examination and measurement conditions can be made uniform (unified). As a result, problems unique to the case of using a photography data can be solved (For example, in consideration of a parameter that can vary as the distance from the first camera 6 to the object 80 changes, references are made uniform in the Z-axis direction, and the like.), external factors (noise and the like) can be reduced as much as possible, or a plurality of subjects can perform measurement under the same environment as much as possible. Therefore, for example, it is possible to accurately know the degree of recovery of the motor function of upper limb or to prevent variation in the examination and measurement states among the subjects. In addition, if the set conditions are visualized or audible by image or voice, the measurement conditions are reliably recognized by the subject 60 (alternatively, external factors that adversely affect the measurement are eliminated), an appropriate measurement environment is prepared, and an accurate measurement result can be obtained.

In the present embodiment, the measurement condition by the condition setter 24 is associated with the measurement environment and the field of view of the subject 60, and the data processor 27 generates audio data that eliminates noise of the measurement environment and/or image data that limits the field of view of the subject 60. For example, in order to suppress unnecessary information from the ear and the eye, the data processor 27 generates audio data for outputting, from the speaker 20, music for canceling surrounding noise as the audio data for eliminating noise in the measurement environment, and generates image data for inserting and hiding a virtual object between the finger of the subject 60 and the surrounding object on the screen 16a, as the image data for limiting the field of view of the subject 60. That is, by playing videos and music on the HMD 50, the subject can relax and take measurements.

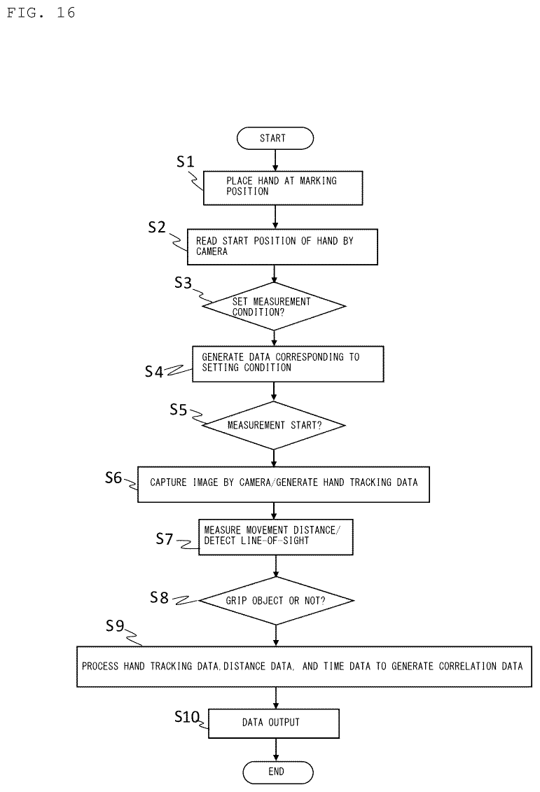

An example of image data generation related to such measurement condition setting is illustrated in FIGS. 14 and 15. FIG. 14 illustrates a state in which a gaze alignment marker 85 is displayed on the screen 16a of the display apparatus 16 of the HMD 50 together with a text display 87 at the time of measurement in which the subject 60 performs the finger tapping motion with the fingers of his/her both hands 63 and 63. In this case, in particular, FIG. 14 (a) illustrates a state in which the entire captured image of first camera 6 including the measurement environment is displayed as it is on screen 16a, and FIG. 14 (b) illustrates a state in which an image (a masking 91 that limits the subject's field of view) that shields the measurement environment except for hand 63 is displayed on screen 16a. The masking 91 is image data that limits the field of view of the subject 60, and the gaze alignment marker 85 is image data corresponding to the measurement condition for concentrating the subject on the measurement, and can also function as image data related to the measurement reference position or image data related to the initial setting.

In addition, FIG. 15 illustrates an image displayed on the screen 16a when the subject 60 performs the examination in which the subject performs the finger tapping motion without looking at his/her hand 63, FIG. 15 (a) is a display image in which the hand 63 of the subject 60 is shielded by a transmission masking 89, and FIG. 15 (b) is a display image in which the contour 76 of the finger serving as a mark of the position where the hand 63 is placed is superimposed on the transmission masking 89 in the display state of FIG. 15 (a).

As described above, in the present embodiment, the display/non-display of the hand portion can be switched according to the measurement application. That is, in the case of measurement in which it is desired to show the motion of the finger, the video captured by the first camera 6 is displayed only for the part of the hand 63 as illustrated in FIG. 14, and in the case of measurement in which the motion of the finger is not shown, the part of the hand 63 is masked as illustrated in FIG. 15 (Alternatively, only the guide (contour) display 76 for alignment is performed.). By preventing the motion of the finger from being shown to the subject 60, it is possible to suppress the influence of the information being seen through the eye on the measurement of the finger tapping motion, and it is possible to align (unify) the measurement conditions.

Note that, in a case where the gaze alignment marker 85 is displayed as illustrated in FIG. 14, it is preferable to perform eye tracking using the second camera 8 as a front camera and the gaze detectors 12 and 14, and to prompt (For example, a voice prompting redoing is made to flow from the speaker 20, or a text prompting re-measurement is displayed on the screen 16a.) re-measurement when it is detected that the line-of-sight of the subject 60 has deviated from the marker 85 during measurement. Alternatively, the angle of the neck of the subject 60 wearing the HMD 50 may be measured using the geomagnetic sensor 25 to urge the subject 60 to face forward (not to tilt the neck downward) rather than toward hand.

FIG. 8 illustrates a state in which the subject 60 moves an arm 64 to move subject's hand 63 to the object 80 placed at a distant position. When the measurement is started in this manner (step S5 in FIG. 16), the imaging data collector 9 (first camera 6) acquires imaging data obtained by capturing the motion of the finger of the subject 60, and the hand tracking data generator 26 generates the chronological hand tracking data from the imaging data by the hand tracking function (Step S6 in FIG. 16 (capturing step, imaging data acquisition step, and hand tracking data generation step)). At the same time, the distance detection sensor 10 measures the chronological movement distance in which the hand 63 moves with the motion of the arm 64 of the subject 60 (the time until the hand 63 is moved to grip the object 80 is also calculated), and the second camera 8 and the gaze detectors 12 and 14 detect the line-of-sight of the eye of the subject 60 (Detect where the subject 60 is looking at at which timing . . . Step S7 of FIG. 16 (movement distance measuring step and gaze detection step)). That is, after the hand 63 placed on the marking 83 is read and recognized via the first camera 6 as described above, the chronological movement distance in which the hand 63 moves with the motion of the arm 64 from the read start position is calculated by the data processor 27 via the distance detection sensor 10.

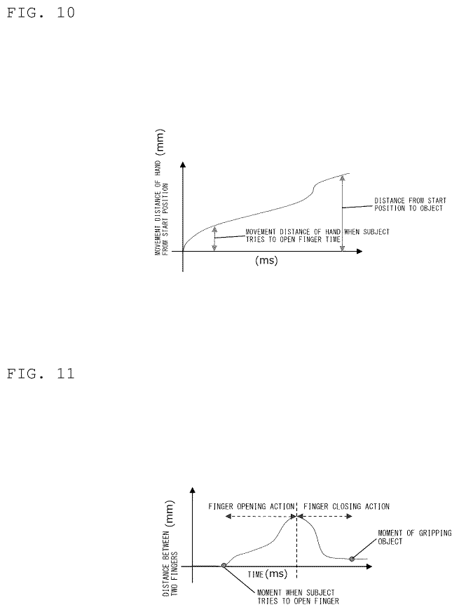

Then, as illustrated in FIG. 9, when the subject 60 grips the object 80 placed at distant position with the hand 63 (step 8 in FIG. 16), the data processor 27 processes the hand tracking data obtained from the hand tracking data generator 26 to generate quantitative data regarding the bending/stretching motion of the finger and/or the opening/closing motion of the two fingers accompanying the motion of the finger joint, and processes the hand tracking data obtained from the hand tracking data generator 26, the distance data and the time data obtained from the distance detection sensor 10, and the line-of-sight data obtained from gaze detectors 12 and 14 via the second camera 8 to generate correlation data obtained by quantifying the correlation between these data (data processing step S9). With such correlation data, the motion of the arm 64 and the opening/closing motion of the finger (this opening/closing motion can be grasped by the hand tracking function) can be evaluated in synchronization (The timing at which the finger is opened and closed can be detected and evaluated in a series of operations in which the arm 64 starts to move and grips the object 80.).

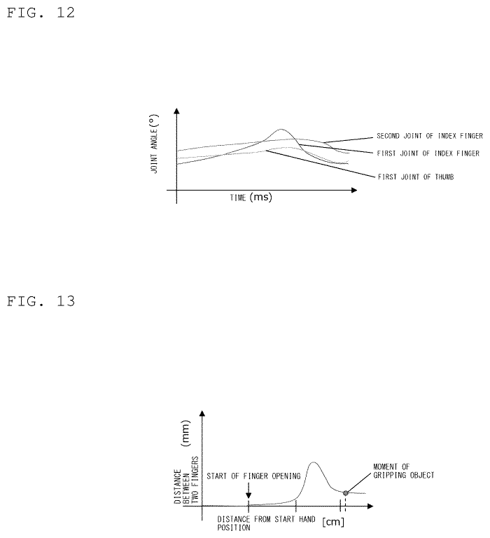

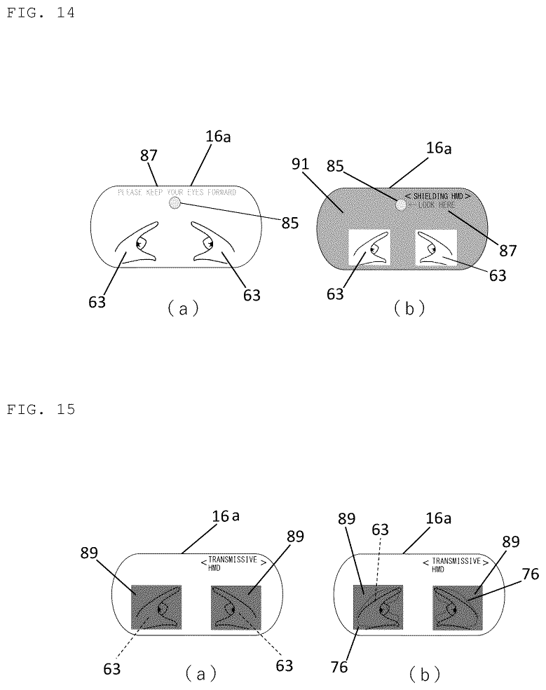

An example of such correlation data is illustrated in FIGS. 10 to 13. FIG. 10 is a graph illustrating a relationship between the movement distance of the hand from the measurement start position to the object to be gripped and the time together with the opening/closing timing of the finger. Such correlation data makes it possible to grasp the movement distance of the hand when the subject tries to open the finger, the distance from the measurement start position to the object, and the time required to grip the object. FIG. 11 is a graph showing a relationship between the distance between two fingers and time. Such correlation data makes it possible to grasp the opening/closing timing of the finger (the moment of gripping the object). FIG. 12 is a graph illustrating a relationship between a joint angle and time obtained from the hand tracking data. Such correlation data makes it possible to grasp a temporal change in the joint angle. FIG. 13 is a graph illustrating a relationship between the distance from the measurement start position and the distance between two fingers. Such correlation data makes it possible to grasp how much the hand has been moved from the measurement start position before the finger has been opened. Note that, in order to be able to evaluate the motion of the finger in synchronization with the motion of the eye detected by the eye tracking of the gaze detectors 12 and 14, for example, correlation data that can display the deviation of the line-of-sight position of the eye with respect to the object to be gripped as a scatter diagram may be generated.

The correlation data as described above can be displayed on the screen 16a together with other data generated by the hand tracking data generator 26 and/or the data processor 27 (output step S10 in FIG. 16).

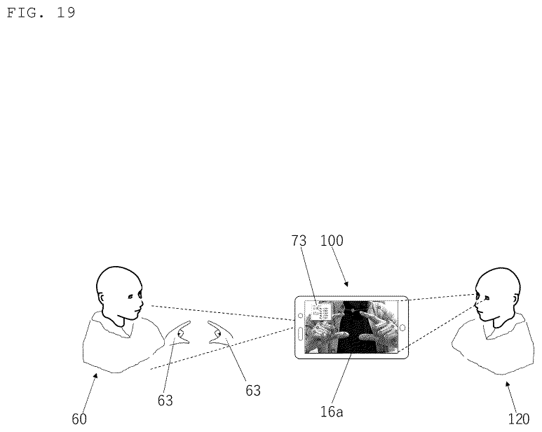

Next, a second embodiment of the present invention will be described with reference to FIGS. 17 to 20. In this embodiment, the measurement processing terminal is configured as a smartphone 100. Note that the basic configuration and operation are the same as those described above with reference to FIGS. 1 and 16 and the like.

FIGS. 17 and 18 illustrate a usage example in which the smartphone 100 is disposed between a face 69 and the hand 63 of the subject 60 and the examination and measurement is performed by the subject alone. FIG. 17 (a) illustrates a state in which the motion of the face and the eye of the subject 60 is captured by the second camera 8 (front camera) of the smartphone 100, and the finger tapping motion (and/or measurement environment) of the subject 60 is tracked and captured by the first camera 6 (rear camera) of the smartphone 100. Note that it is desirable to fix and install the smartphone 100 without holding it with a hand in order to prevent shaking and simultaneously measure both hands. In addition, various types of information related to measurement including a user interface for hand tracking (Since they have been described above, the same reference numerals are given to the drawings and the description thereof is omitted. The same applies to FIGS. 18 to 20.) are displayed on the screen 16a of the display apparatus 16 of the smartphone 100. In addition, the motion of the face and the eye of the subject 60 captured by the second camera 8 is displayed as an inserted image 110 on the screen 16a.

FIG. 17 (b) illustrates how the position and orientation of the hand 63 at the time of measurement are read and registered in advance. The registered information is read before measurement, and the contour guide 76 of the hand is displayed at the position when the information is read. In addition, the display described above regarding the HMD, such as the contour display of the hand and the gaze marker, is similarly performed in the smartphone 100.

FIG. 18 illustrates a state in which online medical examination by a doctor or the like is further performed using the second camera 8 in the state of FIG. 17 (a). In this manner, it is also possible to perform measurement while talking with the doctor displayed as the inserted image 112 on the screen 16a using FaceTime (registered trademark) or the like.

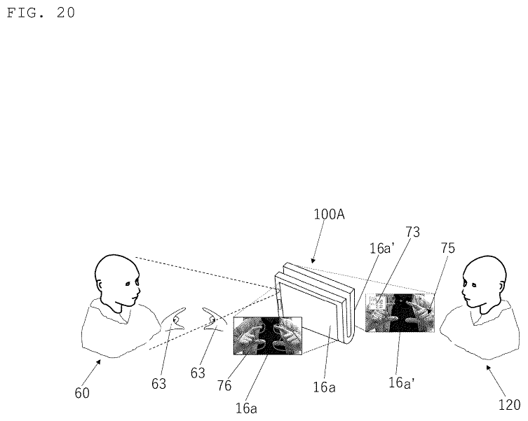

FIGS. 19 and 20 illustrate a usage example in which the smartphone 100 is disposed on the back side of the hand 63 of the subject 60 and the measurer 120 such as a doctor measures the finger tapping motion of the subject 60. Here, FIG. 19 illustrates a state in which the measurer 120 such as the doctor holds the smartphone 100 with his/her hand, and performs tracking and capturing of the motion of the finger together with the upper body of the subject 60 from the front by the first camera 6 of the smartphone 100. The measurer 120 can display and confirm various types of information related to measurement including a user interface for hand tracking on the screen 16a of the smartphone 100. The subject 60 performs the finger tapping motion toward the smartphone 100.

FIG. 20 illustrates a state in which both the subject 60 and the measurer 120 share the examination and measurement image using the smartphone (foldable smartphone) 100A having two display screens 16a and 16a′ that are opened and closed. That is, here, a video captured by the camera on the subject 60 side is also displayed and shared on the screen 16a′ on the measurer 120 side. Specifically, the video captured by the camera is displayed on the subject 60 side, and tracking information and the like are added and displayed on the measurer 120 side. For example, only on the screen on the subject 60 side, the hand 63 is not shown by the masking 89, or the gaze marker 85 and the contour 76 for alignment are displayed. In addition, the video obtained by performing different processing (adding different information) between the subject 60 and the measurer 120, such as displaying the video obtained by superimposing the finger landmark display 72 only on the screen 16a′ on the measurer 120 side with respect to the video captured by the camera, may be displayed. In addition, only the contour may be displayed only on the subject 60 side as a guide during a rehabilitation training.

As described above, according to the first and second embodiments, the chronological hand tracking data can be generated by the hand tracking function from the imaging data obtained by capturing the motion of the finger of the subject 60, and the hand tracking data can be processed to generate quantitative data regarding the bending/stretching motion of the finger and/or the opening/closing motion of the two fingers accompanying the motion of the finger joint. Therefore, the motion of the joint that cannot be recognized by the conventional magnetic sensor type apparatus can be accurately grasped, and the bending/stretching motion of the finger such as a “pinching” motion can be quantitatively evaluated. Unlike the conventional magnetic sensor type apparatus, it is not necessary to attach the sensor to a fingertip.

In addition, according to the first and second embodiments, since the hand tracking data obtained from the hand tracking data generator 26, the distance data and the time data obtained from the distance detection sensor 10, and the line-of-sight data obtained from the gaze detectors 12 and 14 can be processed to generate correlation data obtained by quantifying the correlation between these data, for example, when an arm is moved to grip an object, the motion of the arm and the opening/closing motion of the finger can be quantitatively evaluated at the same time (the interlocking property (relationship) between the motion of the finger and the motion of the arm can be quantitatively evaluated), and furthermore, the interlocking property (relationship) between the motion of the finger and the motion of the eye can be quantitatively evaluated. That is, the finger can be evaluated in synchronization with the motion of another part of the body, and thus the motor function of upper limb can be objectively, finely and accurately evaluated.

Although the embodiments of the present invention have been described above with reference to the drawings, the present invention is not limited to the above-described embodiments and can include various modifications. For example, the above-described embodiments have been described in detail in order to describe the present invention in an easy-to-understand manner, and are not necessarily limited to those having all the described configurations. Further, a part of the configuration of one embodiment can be replaced with the configuration of another embodiment, and the configuration of another embodiment can be added to the configuration of one embodiment. In addition, it is possible to add, delete, and replace other configurations for a part of the configuration of each embodiment.

In addition, some or all of the above-described configurations, functions, processers, processing means, and the like may be realized by hardware, for example, by designing with an integrated circuit. In addition, each of the above-described configurations, functions, and the like may be realized by software by a processor interpreting and executing a program for realizing each function. Information such as a program, a table, and a file for realizing each function may be stored in a recording apparatus such as a memory, a hard disk, and a Solid State Drive (SSD), or a recording medium such as an IC card, an SD card, and a DVD, or may be stored in an apparatus on a communication network.

In addition, the control lines and the information lines indicate what is considered to be necessary for the description, and do not necessarily indicate all the control lines and the information lines on the product. In practice, it may be considered that almost all the configurations are connected to each other.

### REFERENCE SIGNS LIST

6, 8 camera
9 imaging data collector
10 distance detection sensor (movement distance measuring instrument)
12, 14 gaze detector
16 display apparatus (output interface)
20 speaker (output interface)
24 condition setter
26 hand tracking data generator
27 data processor
50 HMD (measurement processing terminal)
100, 100A smartphone (measurement processing terminal)

1. A measurement processing terminal that measures motion of a finger of a subject and processes a measurement result, the measurement processing terminal comprising:
an imaging data collector that collects imaging data obtained by capturing the motion of the finger of the subject;
a hand tracking data generator that implements a hand tracking function of detecting and tracking a position of the finger based on the imaging data, and generates chronological hand tracking data from the imaging data by the hand tracking function; and
a data processor that processes hand tracking data obtained from the hand tracking data generator to generate quantitative data relating to a bending/stretching motion of a finger and/or an opening/closing motion of two fingers with motion of a finger joint.

2. The measurement processing terminal according to claim 1, further comprising:
a movement distance measuring instrument that measures a chronological movement distance of a hand moving with a motion of an arm, wherein
the data processor processes the hand tracking data obtained from the hand tracking data generator and distance data and time data obtained from the movement distance measuring instrument to generate correlation data obtained by quantifying a correlation between the hand tracking data, the distance data and the time data.

3. The measurement processing terminal according to claim 2, further comprising:
a gaze detector that detects a line-of-sight of an eye of the subject, wherein
the data processor processes the hand tracking data obtained from the hand tracking data generator, the distance data and the time data obtained from the movement distance measuring instrument, and line-of-sight data obtained from the gaze detector to generate correlation data obtained by quantifying a correlation between the hand tracking data, the distance data, the time data and the line-of-sight data.

4. The measurement processing terminal according to claim 1, wherein the data processor generates image data related to a measurement reference position and/or a measurement history.

5. The measurement processing terminal according to claim 1, further comprising:
a condition setter that sets a measurement condition, wherein
the data processor generates image data and/or audio data corresponding to the measurement condition set by the condition setter.

6. The measurement processing terminal according to claim 5, wherein the measurement condition is associated with a measurement environment and a field of view of the subject.

7. The measurement processing terminal according to claim 6, wherein the data processor generates audio data for eliminating noise in the measurement environment and/or image data for limiting the field of view of the subject.

8. The measurement processing terminal according to claim 5, wherein the measurement condition is associated with an initial setting of a measurement.

9. The measurement processing terminal according to claim 1, wherein the imaging data collector includes a camera that captures motion of the finger.

10. The measurement processing terminal according to claim 1, further comprising an output interface that outputs data generated by the hand tracking data generator and/or the data processor.

11. A measurement processing terminal that measures motion of a finger of a subject and processes a measurement result, the measurement processing terminal comprising:
a measuring instrument that measures the motion of the finger of the subject;
a data processor that processes measurement data obtained by the measuring instrument; and
a condition setter that sets a measurement condition associated with a measurement environment and a field of view of the subject,
wherein the data processor generates image data and/or audio data corresponding to the measurement condition set by the condition setter.

12. The measurement processing terminal according to claim 11, wherein the data processor generates audio data for eliminating noise in the measurement environment and/or image data for limiting the field of view of the subject.

13. The measurement processing terminal according to claim 11, wherein the measurement condition is associated with an initial setting of a measurement.

14. The measurement processing terminal according to claim 11, further comprising:
an imaging data collector that collects imaging data obtained by capturing the motion of the finger of the subject; and
a hand tracking data generator that implements hand tracking function of detecting and tracking a position of the finger based on the imaging data, and generates chronological hand tracking data from the imaging data by the hand tracking function,
wherein the data processor processes the hand tracking data obtained from the hand tracking data generator to generate quantitative data relating to a bending/stretching motion of the finger and/or an opening/closing motion of two fingers accompanying with motion of a finger joint.

15. The measurement processing terminal according to claim 14, further comprising:
a movement distance measuring instrument that measures a chronological movement distance of a hand moving with a motion of an arm, wherein
the data processor processes the hand tracking data obtained from the hand tracking data generator and distance data and time data obtained from the movement distance measuring instrument to generate correlation data obtained by quantifying a correlation between the hand tracking data, the distance data, and the time data.

16. The measurement processing terminal according to claim 15, further comprising:
a gaze detector that detects a line-of-sight of an eye of the subject, wherein
the data processor processes the hand tracking data obtained from the hand tracking data generator, the distance data and the time data obtained from the movement distance measuring instrument, and a line-of-sight data obtained from the gaze detector to generate a correlation data obtained by quantifying a correlation between the hand tracking data, the distance data, the time data and the line-of-sight data.

17. A measurement processing method for measuring motion of a finger of a subject and processing a measurement result, the measurement processing method comprising:
an imaging data acquisition step of acquiring imaging data obtained by capturing the motion of the finger of the subject;
a hand tracking data generation step of generating a chronological hand tracking data from the imaging data by a hand tracking function of detecting and tracking a position of the finger based on the imaging data; and
a data processing step of processing the hand tracking data obtained from the hand tracking data generation step to generate quantitative data related to a bending/stretching motion of the finger and/or an opening/closing motion of two fingers accompanying a motion of a finger joint.

18. The measurement processing method according to claim 17, further comprising:
a movement distance measuring step of measuring a chronological movement distance in which a hand moves with a motion of the arm, wherein
the data processing step processes the hand tracking data obtained from the hand tracking data generation step and distance data and time data obtained from the movement distance measuring step to generate correlation data obtained by quantifying a correlation between the hand tracking data, the distance data and the time data.

19. The measurement processing method according to claim 18, further comprising:
a gaze detection step of detecting a line-of-sight of an eye of the subject, wherein
the data processing step processes the hand tracking data obtained from the hand tracking data generating step, the distance data and the time data obtained from the movement distance measuring step, and line-of-sight data obtained from the gaze detection step to generate a correlation data obtained by quantifying a correlation between the hand tracking data, the distance data, the time data and the line-of-sight data.

20. The measurement processing method according to claim 17, wherein the data processing step generates image data related to a measurement reference position and/or a measurement history.

21. The measurement processing method according to claim 17, further comprising:
a condition setting step of setting a measurement condition, wherein
the data processing step generates image data and/or audio data corresponding to the measurement condition set in the condition setting step.

22. The measurement processing method according to claim 21, wherein the measurement condition is associated with a measurement environment and a field of view of the subject.

23. The measurement processing method according to claim 22, wherein the data processor generates audio data for eliminating noise in the measurement environment and/or image data for limiting the field of view of the subject.

24. The measurement processing method according to claim 21, wherein the measurement condition is associated with an initial setting of measurement.

25. The measurement processing method according to claim 17, wherein the imaging data acquisition step includes a capturing step of capturing the motion of the finger.

26. The measurement processing method according to claim 17, further comprising:
an output step of outputting data generated by the hand tracking data generation step and/or the data processing step.

27. A computer program for measuring motion of a finger of a subject and processing a measurement result, the computer program causing a computer to execute:
an imaging data acquisition step of acquiring imaging data obtained by capturing the motion of the finger of the subject;
a hand tracking data generation step of generating chronological hand tracking data from the imaging data by a hand tracking function of detecting and tracking a position of the finger based on the imaging data; and
a data processing step of processing the hand tracking data obtained from the hand tracking data generation step to generate quantitative data related to a bending/stretching motion of the finger and/or opening/closing motion of two fingers accompanying a motion of a finger joint.

28. The computer program according to claim 27, further causing the computer to execute:
a movement distance measuring step of measuring a chronological movement distance in which a hand moves with motion of an arm, wherein
the data processing step processes the hand tracking data obtained from the hand tracking data generation step and the distance data and the time data obtained from the movement distance measuring step to generate correlation data obtained by quantifying a correlation between the hand tracking data, the distance data and the time data.

29. The computer program according to claim 28, further causing the computer to execute:
a gaze detection step of detecting a line-of-sight of an eye of the subject, wherein
the data processing step processes the hand tracking data obtained from the hand tracking data generating step, the distance data and the time data obtained from the movement distance measuring step, and a line-of-sight data obtained from the gaze detection step to generate a correlation data obtained by quantifying correlation between the hand tracking data, the distance data, the time data and the line-of-sight data.

30. The computer program according to claim 27, wherein the data processing step generates image data related to a measurement reference position and/or a measurement history.

31. The computer program according to claim 27, further causing the computer to execute:
a condition setting step of setting a measurement condition, wherein
the data processing step generates image data and/or audio data corresponding to the measurement condition set in the condition setting step.

32. The computer program according to claim 31, wherein the measurement condition is associated with a measurement environment and a field of view of the subject.

33. The computer program according to claim 32, wherein the data processor generates audio data for eliminating noise in the measurement environment and/or image data for limiting the field of view of the subject.

34. The computer program according to claim 31, wherein the measurement condition is associated with an initial setting of the measurement.

35. The computer program according to claim 27, wherein the imaging data acquisition step includes a capturing step of capturing the motion of the finger.

36. The computer program according to claim 27, further causing the computer to execute:
an output step of outputting data generated by the hand tracking data generation step and/or the data processing step.
